(19) United States
(12) Patent Application Publication
(10) Pub. No.: US 2004/0142380 A1
(43) Pub. Date: Jul. 22, 2004

(54) Fluorescent labeling reagents and its kit fabricated by B-phycoerythrin and its derivatives

(75) Inventor: Chuang-Chun Chiueh, Taipei (TW)

Correspondence Address: ARENT FOX KINTNER PLOTKIN & KAHN, PLLC, Suite 400, 1050 Connecticut Avenue, N.W., Washington, DC, 20036-5339, US

(73) Assignee: Far East Biotec Co., Ltd.

(21) Appl. No.: 10/393,996

(22) Filed: Mar. 24, 2003

(30) Foreign Application Priority Data
Jan. 17, 2003 (TW) 92101040

(51) Int. Cl. G01N 33/53

(52) U.S. Cl. 435/7.1

(57) ABSTRACT

The present invention provides for several fluorescent labeling reagents and its kits that comprises B-phycoerythrin and other chemical fluorescent reagents that form a range of various fluorescence emission spectrums. The invented fluorescent labeling reagent can be used by itself or in conjunction with other kinds of reagents. When used by itself, one light source can output many kinds of fluorescent lights, making it useful in immunoassay, flow cytometry, and fluorescent light analysis.

### BACKGROUND OF THE INVENTION

1. Field of the Invention

The present invention provides for several fluorescent labeling reagents and its kit which were fabricated by conjugated B-phycoerythrin and other chemical fluorescent reagents that forms a series fluorescent labeling reagents with various fluorescent emission spectrums.

2. Description of Related Arts

Currently there are many different kinds of labeling reagents such as fluorescent labels or dye that are being widely used in various biomolecule detection analysis, such as cell differentiation, diagnostic analysis, fluorescence microscope and immunocyte chemical positioning markers. Some of those analytical techniques were improved due to advances in immunoassay technologies, such as electron image microscope, and flow cytometers, that allows for usage of fluorescent labeling antibody, DNA probe, biological analogs, cells, polymers, etc., which were techniques growing very rapidly.

In biology and medicine today, fluorescent immunoassay has been one of the frequently used techniques that can be used in immunoassay analysis, disease screening, laboratory experiment, etc. Currently there are many different kinds of fluorescent labeling reagents that emit different colors, but the common problems are low molecular stability, small fluorescent light output, shading of fluorescent lights, and scattering of excited light sources, making those reagents hard to be used simultaneously. Thus, any future development of new fluorescent labeling reagent should fulfill at least one of these criteria: increased fluorescence output, improved light stability, lowered non-specific binding and selected the excited and emitted light that are more suitable for the light source of the spectrometers and detectors. For these reasons, this invention offers a fluorescent labeling reagent and its kits that use B-phycoerythrin and its derivatives to solve problems found in prior art.

### SUMMARY OF THE INVENTION

To solve the problem described above, this invention put forth the fluorescent labeling reagents that conjugate of a B-phycoerythrin and chemical fluorescent reagents. The chemical fluorescent reagent absorbs energy from B-phycoerythrin, resulting in excitation of said chemical fluorescent reagent to emit the fluorescence. Another purpose in this invention is to provide a kit for using in a multiplex fluorescent detection assays, comprising B-phycoerythrin and at least one derivative of conjugated B-phycoerythrin and a chemical fluorescent reagent. The chemical fluorescent reagent absorbs energy from B-phycoerythrin, resulting in excitation of said chemical fluorescent reagent to emit the fluorescence.

### BRIEF DESCRIPTION OF DRAWINGS

FIG. 1 shows the fluorescence spectrum of the B-phycoerythrin.

FIG. 2 shows the fluorescence spectrum of the four kinds of the fluorescent labeling reagents of this invention.

### DETAILED DESCRIPTION OF THE INEVNTION

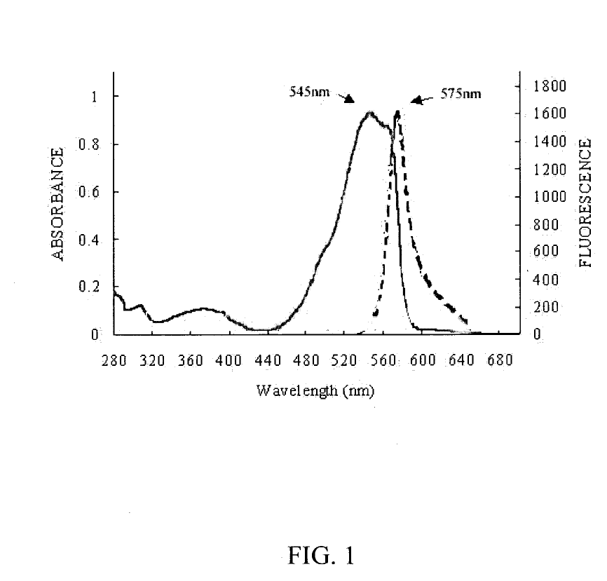

B-phycoerythrin is different from R-phycoerythrin that was discovered in the past and the differences are described in chart 1. B-phycoerythrin's main characteristics are as follows: Its alpha (α), beta (β), and gamma (γ) subunits usually form into a hexamer ((αβ)₆γ) shape. It has a molecular weight of 240,000 Daltons. Its light absorption range falls within the visible light area, as described in FIG. 1. Its light absorption spectrum's peak is at 545 nm, and around 498 nm there is a smaller peak. Its fluorescence emission range falls within the visible light range, as the light emission spectrum reveals in FIG. 1 (appears in dotted line). Its fluorescence emission spectrum peaks at 575 nm. Its fluorescence intensity is strongly stable. Environmental impact caused by pH value, temperature and existence of other biomolecules is quite small. Finally, its excitation light wavelength is best at 543 nm, which results in fluorescence quantum yield at 0.98

Growing B-phycoerythrin is quite difficult, due to limited supply of proteins, resulting in small amount of consumer of such item. However, B-phycoerythrin's ability to generate fluorescence (i.e., its quantum exchange rate) is very high, making it an excellent fluorescent labeling reagent by nature. Under the appropriate conditions, B-phycoerythrin can also by itself conduct effective energy transmission (due to its high quantum transfer rate). It can form derivatives by conjugating with a chemical fluorescent reagent. The derivative uses its own ability to absorb lights and transfer energy with high efficiency to excite the chemical fluorescent reagent to emit fluorescent light with long wavelengths. Hence, a positive attribute in the derivative is in its stock shift being clearly increased, resulting in lowering of background values. Furthermore, since the chemical fluorescent reagent was bound to the B-phycoerythrin, the fluorescence they together generated cannot be easily shielded.

TABLE 1
Comparison of Two Kinds of Phycoerythrin

| | B-phycoerythrin | | R-phycoerythrin | |
|---|---|---|---|---|
| Molecular Mass | 240 | KD | 264 | KD |
| Greatest Absorption | 545 | nm | 566 | nm |
| Wavelength | | | | |
| Best laser Excited Source | 543 | nm | 488 | nm |
| Greatest Fluorescence | 575 | nm | 575 | nm |
| Wavelength | | | | |
| Extinction Coefficient | 2.41 × 10⁶ cm⁻¹ · M⁻¹ | | 1.96 × 10⁶ cm⁻¹ · M⁻¹ | |
| Fluorescence Exchange | 0.98 | | 0.84 | |
| Rate | | | | |

Today, laser light is the most frequent form of excitation light source for fluorescenec spectrometers such as flow cytometer, fluorescence immunoassay, and conjugate focus microscope. Previously, to select a combination of different fluorescent emission lights, because each fluorescence labeling reagent's excitation light source is different, requires the addition of many laser light sources, making the building such spectrometer very expensive. As an improvement, the present invention uses B-phycoerythrin derivatives (created by B-phycoerythrin conjugating with a chemical fluorescent reagent) in combination. Such combination uses excitable B-phycoerythrin as the one light excitation source, and can simultaneously emit multicolored fluorescence, making this invention a better and economical method for building multicolored fluorescent light spectrometer.

When using many kinds of fluorescence at the same time, one should further consider possible technical limitations with respect to mutual differentiation and interference among the lights. Previously, fluorescence spectrometer that uses laser light as the excitation light source can differentiate fluorescence wavelengths with 20 nm precision. Thus, this invention focuses on this limitation and offers B-phycoerythrin derivatives that cause fluorescent lights to evenly fall within the visible light range and to separate from each other at about 30 nm apart. Of course, this invention uses B-phycoerythrin and its derivative to create fluorescentlabeling reagents, using any excitation light source that can excite B-phycoerythrin.

This invention uses chemical fluorescent reagent that can easily combine with B-phycoerythrin and transfer energy absorbed by B-phycoerythrin to a chemical fluorescent reagent to excite the chemical fluorescent reagent to emit fluorescent light. The chemical fluorescent reagent mentioned above can be Texas red, Cy5, or Cy5.5.

Manufactured according to the present invention are fluorescent labeling reagent kit as described in table 2.

TABLE Two
Fluorescent Labeling Reagent Kit

| Name of Fluorescent | Chemical Fluorescent | Fluorescence Emission |
|---|---|---|
| Labeling Reagent | Labeling Reagent | Wavelength (nm) |
| BPE | None | 570-590 nm |
| BPE 620 | Texas Red | 610-630 nm |
| BPE 660 | Cy5 | 650-680 nm |
| BPE 700 | Cy5.5 | 680-700 nm |

The invented multicolored fluorescent labeling reagent kit's labeling reagent can be practiced by simultaneously using only one or multiple labeling reagents. When using one wavelength of an excitation light source, one can at the same time detect emission wavelengths for various fluorescent lights. Therefore, such invention allows manufacturers to save overhead costs in building spectrometers and can solve problems of mutual interference among various sources of lights. The wavelength of the excitation light using for the above mentioned multicolored fluorescent labeling reagent kit can range from 488 nm to 633 nm, with the most preferred excitation light source wavelength at 543 nm.

Moreover, the invented fluorescent labeling reagents that can be combined with proteins, such as antigens or antibody, which can be used for immunoassay, flow cytometer or fluorescence analysis.

Best Mode of Present Invention:

(1) Preparation of B-phycoerythrin:

B-phycoerythrin compositions are specified as follows: (i) Light absorption spectrum values are A_{545 nm}/A_{280 nm}≧5.5, A_{565 nm}/A_{545 nm}≧0.95, A_{620 nm}/A_{545 nm} ≦0.005. (ii) Protein purity is above 98%. (iii) Preparation are to be stored in a solution comprising of 150 mM of Sodium phosphate buffer at pH7.0, 60% ammonium sulfate, 1 mM of Sodium Ethylenediaminetetra-acetate or EDTA, and 1 mM of sodium azide, at 4° C.

(2) Preparation of Multicolored Fluorescent Labeling Reagent:

BPE 620 is created by B-phycoerythrin conjugated with Texas Red (Sigma). Replace B-phycoerythrin which is stored in 60% liquid Sodium phosphate buffer into a solution of alkaline buffer by using a dialysis method. Then, add Texas Red which dissolved in DMSO. The molar ratio of the B-phycoerythrin and Texas Red is 1:1 and the concentration range of the B-phycoerythrin is from 0.1 to 1 mM. This preparation is to be stored in a dark indoor space for at least 4 hours. Then, use molecular sieving column to purify the BPE620.

BPE 660 is formed by B-phycoerythrin conjugating with Cy5 (Amersham), while BPE 700 is formed by B-phycoerythrin conjugating to Cy5.5 (Amersham). Preparation for either two fluorescent labeling reagents is similar: Replace B-phycoerythrin which is stored in 60% liquid Sodium phosphate buffer into a solution of alkaline buffer by using a dialysis method. Followed by adding the Cy chemical fluorescent reagent (such as Cy5 or Cy5.5) dissolved in DMSO. The molar ratio of the B-phycoerythrin and Texas Red is 1:1 and the concentration range of the B-phycoerythrin is from 0.1-1 mM. The mixture is allowed to undergo reaction in a dark indoor environment for 1 hour. Finally one is to use molecular sieving column to purify the BPE660 and BPE700.

(3) The Fluorescence Spectrums of the Multicolored Fluorescent Labeling Reagent

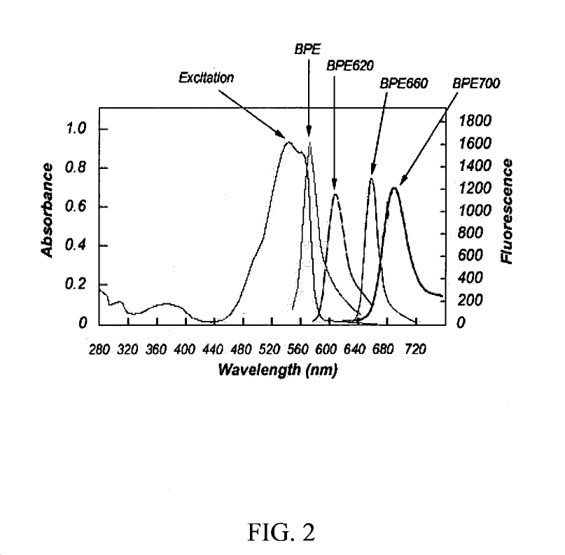

One first take a mixture of B-phycoerythrin and BPE 620, BPE 660, BPE 700 created in step (2) and dissolve the mixture in 150 mM liquid Sodium phosphate buffer at pH7.0. Next, use 543 nm laser light to excite the mixture, a fluorescence spectrum of 4 kinds of fluorescent labeling reagent are obtained as shown in FIG. 2, which reveals the fluorescence emission wavelength from top to bottom: BPE at 575 nm, BPE620 at 613 nm, BPE 660 at 667 nm and BPE 700 at 695 nm.

What is claimed is:

1. A fluorescent labeling reagent, comprising a conjugate of a B-phycoerythrin and a chemical fluorescent reagent, wherein said chemical fluorescent reagent absorbs energy from B-phycoerythrin, resulting in excitation of said chemical fluorescent reagent to emit the fluorescence

2. The fluorescent labeling reagent according to claim 1, wherein said chemical fluorescent reagent comprises Texas red, Cy5, and Cy5.5.

3. The fluorescent labeling reagent according to claim 1, wherein said B-phycoerythrin excitation wavelength is within a range of 488 nm to 633 nm.

4. The fluorescent labeling reagent according to claim 3, wherein said wavelength is preferred to be 543 nm.

5. The fluorescent labeling reagent according to claim 1, wherein said B-phycoerythrin fluorescent emission wavelength is within a range of 570 nm to 590 nm.

6. The fluorescent labeling reagent according to claim 1, wherein said B-phycoerythrin conjugates to Texas red (BPE 620), having a fluorescent emission wavelength within a range of 610 to 630 nm.

7. The fluorescent labeling reagent according to claim 1, wherein said B-phycoerythrin conjugates to Cy5 (BPE660), having a fluorescent emission wavelength within a range of 650 to 680 nm.

8. The fluorescent labeling reagent according to claim 1, wherein said B-phycoerythrin conjugates to Cy5.5 (BPE700), having a fluorescent emission wavelength within a range of 680 to 700 nm.

9. A kit for using in a multiplex fluorescent detection assay, comprising:
a B-phycoerythrin; and
at least one derivative of conjugated B-phycoerythrin and a chemical fluorescent reagent, wherein said chemical fluorescent reagent absorbs energy from B-phycoerythrin, resulting in excitation of said chemical fluorescent reagent to emit the fluorescence.

10. The kit according to claim 9, wherein said chemical fluorescent reagent comprises Texas red, Cy5, and Cy5.5.

11. The kit according to claim 9, comprising:
a B-phycoerythrin; and
a conjugate of B-phycoerythrin and Texas red (BPE620);
a conjugate of B-phycoerythrin and Cy5 (BPE660); and
a conjugate of B-phycoerythrin and Cy5.5 (BPE700).

12. The kit according to claim 9, wherein said excitation wavelength is within a range from 488 nm to 633 nm.

13. The kit according to claim 12, wherein said excitation wavelength is preferably 543 nm.

14. The kit according to claim 11, wherein B-phycoerythrin fluorescent emission wavelength is within the range of 570 to 590 nm.

15. The kit according to claim 11, wherein said conjugate of B-phycoerythrin and Texas red (BPE620) has a fluorescent emission wavelength within the range of 610 to 630 nm.

16. The kit according to claim 11, wherein said conjugate of B-phycoerythrin and Cy5 (BPE660) has a fluorescent emission wavelength within the range of 650 to 680 nm.

17. The kit according to claim 11, wherein said conjugate of B-phycoerythrin and Cy5.5 (BPE700) has a wavelength within the range of 680 to 700 nm.

18. The kit according to claim 9, wherein said fluorescent labeling reagents can be used only one or conjunction with other fluorescent labeling reagents.

19. The fluorescent labeling reagent according to claim 1, wherein said fluorescent labeling reagent further can conjugate with proteins such as antigen or antibody to be used in immunoassay, flow cytometry, or fluorescent light analysis.
